(19) United States
(12) Patent Application Publication
(10) Pub. No.: US 2026/0013768 A1
(43) Pub. Date: Jan. 15, 2026

(54) MULTI-ELECTRODE ARRAY DEVICE

(71) Applicant: DYCONEX AG, Bassersdorf (CH)

(72) Inventors: Eckardt BIHLER, Uitikon (CH); Hubert ZIMMERMANN, Staufen (CH)

(73) Assignee: DYCONEX AG, Bassersdorf (CH)

(21) Appl. No.: 18/881,287

(22) PCT Filed: Jun. 8, 2023
(86) PCT No.: PCT/EP2023/065404

§ 371 Date: Jan. 5, 2025

(30) Foreign Application Priority Data
Jul. 6, 2022 (EP) 22183340.3

(51) Int. Cl. A61B 5/262; A61B 5/00; A61B 5/263; A61B 5/279

(52) U.S. Cl. CPC A61B 5/262; A61B 5/263; A61B 5/279; A61B 5/685; A61B 2560/0468; A61B 2562/046; A61B 2562/125; A61B 2562/227

(57) ABSTRACT

A multi-electrode array device (1) comprises a substrate (10) having a substrate body (100), and a multiplicity of electrodes formed by a multiplicity of needle elements (120) arranged on said substrate body (100) and spaced with respect to each other along a plane (P). The needle elements (120) are formed from a metal layer (12) arranged on said substrate body (100), each needle element (120) comprising a contact section (121) extending along said plane (P) and a needle section (122) extending from said contact section (121) and having a tip (123), wherein said needle section (122) is bent with respect to said contact section (121) such that the needle section (122) with its tip (123) protrudes from said plane (P).

The invention relates to a multi-electrode array device according to the preamble of claim 1 and to a method for fabricating a multi-electrode array device.

A multi-electrode array device of this kind comprises a substrate having a substrate body, and a multiplicity of electrodes formed by a multiplicity of needle elements arranged on said substrate body and spaced with respect to each other along a plane.

A multi-electrode array device of the kind concerned herein for example is applicable for in vivo or in vitro neuronal action potential recordings. A multi-electrode array device of the type concerned herein may for example be used during open brain surgery to record electrical signals on brain tissue and for this may be placed on the somatosensory cortex of the brain of a patient. A multi-electrode array device of the kind concerned herein likewise may be designed as an implantable device for (permanent) implantation in a patient.

A multi-electrode array device comprises a multiplicity of electrodes formed by a multiplicity of microscale needle elements which project from a substrate body and may be brought into engagement with brain tissue to allow for an in vivo or in vitro recording of neuronal action potentials. The microscale needle elements herein may be brought in contact with neuronal cells, such that the needle elements may pick up neuronal signals and hence allow for a recording of signal patterns on neuronal cell tissue.

Commonly used micro-electrode arrays for in vivo applications are typically silicon-based, i.e. comprise a plurality of conductive silicon needles. One example is the Utah electrode array, which is wide commercially used and considered so far as industry benchmark for recording large population of neurons.

It is an object of the instant invention to provide a multi-electrode array device and a method for fabricating a multi-electrode array device which allow in an easy, cost-efficient, yet reliable manner to produce an array of needle elements to form electrodes for recording neuronal signals.

This object is achieved by means of the subject matter of claim 1.

Accordingly, a multi-electrode array device comprises a substrate having a substrate body and a multiplicity of electrodes formed by a multiplicity of needle elements arranged on the substrate body and spaced with respect to each other along a plane. The needle elements are formed from a metal layer arranged on the substrate body, each needle element comprising a contact section extending along the plane and a needle section extending from the contact section and having a tip, wherein the needle section is bent with respect to the contact section such that the needle section with its tip protrudes from the plane.

The multi-electrode array device is formed from a substrate which carries a metal layer. Needle elements herein are formed from the metal layer on the substrate, the needle elements hence being carried by the substrate and being spaced with respect to each other along a plane along which the (planar) substrate extends. Each needle element comprises a contact section which is placed on the substrate body of the substrate and is formed from the metal layer to extend along the plane of the substrate body. A needle section protrudes from the contact section of each particular needle element such that the needle section extends from the contact section and, in an operative state, is oriented to emerge from the plane of the substrate in order to protrude from the substrate to be exposed towards the outside for engaging with tissue during operation of the multi-electrode array device. The needle sections, at an end opposite to the contact section, form tips which point away from the substrate and are configured to engage with tissue, in particular brain tissue, to record neuronal action potentials in in vivo or in vitro applications of the multi-electrode array device.

As the needle elements are formed from a metal layer arranged on the substrate body of the substrate, fabrication of the multi-electrode array device becomes easy and cost-efficient, yet at the same time reliable. In particular, common fabrication techniques as known from the fabrication of semiconductor devices are applicable to achieve a structuring of the metal layer on the substrate, upon which the needle sections of the needle elements are bent to assume their protruding, bent shape to form an array of regularly or irregularly spaced needle elements for engaging with tissue.

The electrodes formed by the needle sections of the needle elements may in particular have a microscopic scale. Each needle section for example may have a length in between 10 μm to 100 μm, for example between 20 μm to 30 μm, a width in between 1 μm and 50 μm, for example between 5 μm and 15 μm, and a thickness (corresponding to the thickness of the metal layer on the substrate) between 1 μm and 50 μm, for example between 1 μm and 10 μm.

The metal layer on the substrate may in particular be made of a gold material, in particular pure gold. The metal layer may have a thickness in between 1 μm to 50 μm.

The substrate body may have a thickness in between 10 μm to 100 μm.

In one embodiment, the substrate body forms a surface extending along the plane, the needle elements being arranged on the surface such that the contact sections are placed on the surface and the needle sections protrude from the surface. The contact sections form planar, for example circular, sections which rest on the surface of the substrate body. The needle sections extend from the contact sections such that the needle sections are oriented at an angle with respect to the plane of the substrate, for example at an angle in between 45° to 90°, for example between 60° and 90°. The needle sections are formed from the metal layer of the substrate and hence are integral with the contact sections, the needle sections being bent with respect to the contact sections to point out of the plane of the surface of the substrate.

In one embodiment, the multi-electrode array device comprises a first cover layer covering the surface of the substrate body and at least the contact sections such that the contact sections are electrically insulated towards the outside. The cover layer is made from an electrically insulating material, the cover layer extending across the substrate such that the contact sections of the needle elements are embedded in between the cover layer and the substrate body, wherein the needle sections with their tips protrude from the first cover layer and hence are exposed towards the outside.

The first cover layer may also cover a portion of the needle section of each needle element such that only the tip of each needle section is exposed towards the outside and may come into contact with tissue when the multi-electrode array device is placed on tissue. By electrically insulating a substantial portion of each needle section, neuronal action potentials may be picked up via the tips of the individual needle sections in a localized fashion, hence allowing for recordings of distinguished neuronal signals on neuronal cells.

In one embodiment, the substrate body is formed from a thermoplastic material. The substrate body may for example be formed from a liquid crystal polymer (LCP) material. In another embodiment, the substrate body may be formed from PEEK, PTFE, FEP or another thermoplastic biocompatible material.

Within the meaning of the present invention, the term “liquid crystal polymer” is used in the meaning known to and commonly used by a person skilled in the art. A “liquid crystal polymer” refers in particular to an aromatic polymer, which has highly ordered or crystalline regions in the molten state or in solution. Non-limiting examples include aromatic polyamides such as aramid (Kevlar) and aromatic polyesters of hydroxybenzoic acid, such as a polycondensate of 4-hydroxybenzoic acid and 6-hydroxynaphthalene-2-carboxylic acid

### (Vectran).

In one embodiment, the substrate body material comprises a comparatively high melting point, beneficially a melting point at a temperature larger than 200° C., for example larger than 250° C.

The first cover layer arranged on the substrate body to cover the surface of the substrate body and the contact sections arranged thereon likewise may be fabricated from a thermoplastic material, for example a liquid crystal polymer (LCP) material, PEEK (polyether ether ketone), PTFE (polytetrafluoroethylene), FEP (fluorinated ethylene propylene) or another thermoplastic biocompatible material.

The first cover layer may be formed from the same material as the substrate body, or from a different material.

The first cover layer may have a thickness in between 10 μm to 100 μm.

In one embodiment, the multi-electrode array device comprises an electronics device. Needle elements are arranged on a first side of the substrate body, and the semiconductor device is arranged on a second side of the substrate body opposite to the first side. The electronics device is electrically connected to the contact sections of the needle elements by an arrangement of electrical vias extending through the substrate body. In particular, in one embodiment, the substrate may comprise a first metal layer on the first side of the substrate body, the first metal layer forming the needle elements. In addition, the substrate may comprise a second metal layer arranged on the second side of the substrate body, the second metal layer forming contact pads on the second side of the substrate body for contacting the electronics device arranged on the second side of the substrate body. The contact sections of the needle elements on the first side of the substrate body and the contact pads on the second side of the substrate body are connected by means of electrical vias, such that through-contacts are formed on the substrate body allowing an electrical connection between the needle elements on the first side of the substrate body and the electronics device placed on the second side of the substrate body.

The second metal layer on the second side of the substrate body may be formed by a gold layer or by a copper layer or by another electrically conductive metal material. Likewise, vias may be formed from a gold material or from a copper material, for example a gold plating or a copper plating.

In one embodiment, the micro-electrode array comprises a form element being arranged on the second side of the substrate body, wherein the form element comprises a multiplicity of protrusion members abutting the needle sections of the multiplicity of needle elements. The form element may for example be provided as an injection molded part for example made from a thermoplastic material, such as for example a liquid crystal polymer (LCP) material, PEEK, PTFE, FEP or another thermoplastic biocompatible material. The form element may comprise a body portion, the protrusion members protruding from the body portion to engage with the needle sections when the form element is arranged on the substrate body. In that the form element comprises a number of protrusion elements corresponding to the number of needle elements, all needle sections may be abutted. The protrusion members of the form element serve to act onto the needle sections such that the needle sections are bent with respect to the contact sections to cause the needle sections to protrude from the substrate body in a direction pointing out of the plane of the substrate body.

In one embodiment, substrate body comprises openings being formed in the substrate body such that each needle section projects into a space aligned with a corresponding opening. Each needle section hence is associated with an opening within the substrate body, the opening extending through the substrate body from a first side (on which the needle elements are arranged) towards a second side opposite the first side. The form element may be arranged on the substrate body such that the protrusion members are introduced into the openings in the substrate body to abut the needle sections. The openings may for example have a circular cross-section. The openings may have a diameter in a range for example in between 100 μm to 10 mm.

In one embodiment, the body portion of the form element comprises openings, wherein the form element is arranged on the substrate body such the openings are aligned with the arrangement of electrical vias extending through the substrate body and/or the contact pads on the second side of the substrate body for contacting the electronics device arranged on the second side of the substrate body, wherein particularly the electronics device is arranged on the form element such that the electronics device is electrically contacted to the arrangement of electrical vias and/or to the contact pads through the body portion of the form element.

In one embodiment, the electronics device is encapsulated, on the second side of the substrate body and/or the form element, within electrically insulating material of a second cover layer on the second side of the substrate body. The electronics device hence is embedded within the substrate material of the multi-electrode array device, such that the electronics device is electrically insulated towards the outside, wherein connection lines for example for an electrical power supply and for a signal transmission may extend through the material of the second cover layer to provide for a connection of the electronics device within the encapsulating material of the multi-electrode array device.

The second cover layer beneficially is made from a thermoplastic material, for example a liquid crystal polymer (LCP) material, PEEK, PTFE, FEP or another thermoplastic biocompatible material. The second cover layer herein may be made up from different sub-layers, wherein an inner sub-layer may be made from a thermoplastic material having a comparatively low melting point, for example a melting point at a temperature in between 100° C. and 200° C. An outer sub-layer in contrast may have a melting point at a significantly higher temperature, for example a temperature larger than 200° C., for example larger than 250° C.

In another aspect, a method for fabricating a multi-electrode array device comprises: providing a substrate having a substrate body, and providing a multiplicity of electrode formed by a multiplicity of needle elements on said substrate body such that the needle elements are spaced with respect to each other along a plane. Herein, providing the multiplicity of electrodes formed by the multiplicity of needle elements includes: forming the needle elements from a metal layer arranged on said substrate body such that each needle element comprises a contact section extending along said plane and a needle section extending from said contact section and having a tip, wherein said needle section is bent with respect to said contact section such that the needle section with its tip protrudes from said plane.

The advantages and advantageous embodiments as described above for the multi-electrode array device equally apply also to the method, such that it shall be referred to the above in this respect. Likewise, the following embodiments as described with reference to the method equally apply also to the multi-electrode array device.

Within the method for fabricating the multi-electrode array device, the needle elements are formed from a metal layer arranged on the substrate body of the substrate. The needle elements hence are formed from a structured metal layer formed on the substrate body, wherein contact sections rest on the substrate body and extend along a plane along which the metal layer extends. During the fabrication, the needle sections are bent with respect to the contact sections such that the needle sections protrude from the plane and hence from the substrate body to point, with their tips, towards the outside to be exposed towards the outside and to come into electrical contact with tissue during operation of the multi-electrode array device.

The forming of the needle elements from the metal layer on the substrate body may employ common semiconductor fabrication techniques, such as plasma or chemical etching techniques or laser ablation techniques.

In one embodiment, the needle elements are formed from the metal layer by forming the contact sections and the needle sections to commonly extend along the plane and to subsequently bent the needle sections with respect to the contact sections such that the needle sections protrude from the plane. In an initial state, hence, for each needle element a planar structure of a contact section and a needle section extending from the contact section in the plane of the metal layer is formed. In a subsequent step, the needle sections are bent with respect to the contact sections and are hence formed to protrude from the contact sections to point out of the plane, for example at an angle in between 45° to 90°, for example between 60° and 90°. The needle sections with their tips hence point away from the substrate body towards the outside and, by protruding from the substrate body, may be brought into engagement with tissue to electrically contact cell tissue during operation of the multi-electrode array device.

The bending of the needle sections may take place in a single step such that all needle sections are bent at the same time. For this, in one embodiment, a form element may be placed on the substrate body, the form element comprising a multiplicity of protrusion members to act onto the needle sections for bending the needle sections with respect to the contact sections. The form element may for example be provided as an injection molded part for example made from a thermoplastic material, such as for example a liquid crystal polymer (LCP) material, PEEK, PTFE, FEP or another thermoplastic biocompatible material. The form element may comprise a body portion, the protrusion members protruding from the body portion to engage with the needle sections when placing the form element on the substrate body. In that the form element comprises a number of protrusion elements corresponding to the number of needle elements, all needle sections may be bent with respect to the contact sections in a single step by the placement of the form element on the substrate body and the action of the protrusion members of the form element on the needle sections.

The protrusion members of the form element serve to act onto the needle sections such that the needle sections are bent with respect to the contact sections to cause the needle sections to protrude from the substrate body in a direction pointing out of the plane of the substrate body. Prior to placing the form element on the substrate body for bending the needle sections, herein, in one embodiment openings are formed on the substrate body such that each needle section projects into a space aligned with a corresponding opening. Each needle section hence is associated with an opening within the substrate body, the opening extending through the substrate body from a first side (on which the needle elements are arranged) towards a second side opposite the first side. By forming the openings, substrate material is removed in the region of the needle sections, the needle sections hence projecting from the contact sections without supporting substrate material underneath. To effect the bending of the needle sections, then, the form element may be placed on the substrate body such that the protrusion members are introduced into the openings in the substrate body to act onto the needle sections.

The openings may for example have a circular cross-section. The openings may have a diameter in a range for example in between 100 μm to 10 mm.

For forming the openings in the substrate body, a structuring technique such as laser ablation, plasma etching or chemical etching may be employed.

The form element, for bending the needle sections, may be placed on the substrate body such that the form element comes to lie on a side of the substrate body opposite of the side on which the needle elements are formed. The needle elements hence are formed and arranged on the first side of the substrate body, and the form element is placed on the second side of the substrate body such that the protrusion members of the form element extend through the openings in the substrate body to act onto the needle sections on the first side of the substrate body.

The bending of the needle sections by using the form element herein may take place at an increased temperature, for example in between 100° C. to 200° C., for example by blowing heated air towards the substrate body with the needle sections arranged thereon. By heating the needle sections, a bending and hence a plastic deformation of the microscopic needle elements may be facilitated in that the needle sections may easily adapt to the shaping by the protrusion members.

The protrusion members are formed to act onto the needle sections to deflect the needle sections out of the plane of the contact sections. The protrusion members herein may be shaped such that each protrusion member receives a portion of a corresponding needle section within such that the protrusion member, in an operative state of the multi-electrode array device, provides for an electrical insulation on the needle section (while leaving the tip of the needle section exposed towards the outside).

In one embodiment, prior to or after placing the form element on the substrate body for bending the needle sections, a first cover layer is formed on the substrate body to at least cover the contact sections of the needle elements. The first cover layer is formed on the first side of the substrate body, hence at a side of the substrate body opposite to the form element. The cover layer is formed from an electrically insulating material, in particular from a thermoplastic material, such as for example a liquid crystal polymer (LCP) material, PEEK, PTFE, FEP or another thermoplastic biocompatible material.

The first cover layer may have a thickness in between 10 μm to 100 μm. Similarly, the substrate body may have a thickness in between the 10 μm to 100 μm, wherein the thickness of the first cover layer and the substrate body may be equal or may be different from one another.

The first cover layer may be placed on the substrate body prior to forming the openings within the substrate body for insertion of the protrusion members of the form element, or after the openings in the substrate body are formed. For forming the openings in the substrate body and/or in the first cover layer, structuring techniques such as laser ablation, plasma etching or chemical etching may be employed. For this, the first cover layer may in particular be a photo-structurable polymer, such as a photoresist material.

In addition to providing for an electrically insulating covering for the contact sections of the needle elements towards the outside, the first cover layer may cover a portion of each needle section such that (only) the tips of the needle sections are exposed towards the outside and may come into electrical contact with neuronal cell tissue.

In one embodiment, for bending the needle sections, the form element is placed on the substrate body along a placement direction (perpendicular to the plane of the substrate body along which the needle elements are spaced) such that the substrate body is arranged on a first side of the form element. Herein, after placing the form element on the substrate body for bending the needle sections, an electronics device may be placed on a second side of the form element opposite to the first side of the form element and is electrically connected to the contact sections of the needle elements.

The electronics device for example may comprise circuitry for providing for a processing of signals received via the array of needle elements forming electrodes for electrically contacting tissue. The electronics device for example may comprise circuitry for amplifying and digitizing received signals and for multiplexing signals for transmission to an external processing circuitry. The electronics device hence may provide for a preprocessing of signals for a transmission to external processing circuitry for a further processing.

The electronics device may for example be provided as a semiconductor device, for example as an ASIC chip.

The electronics device may be electrically connected to the contact sections of the needle elements by a second metal layer on the second side of the substrate body, the second metal layer forming contact pads associated with the contact sections of the needle elements and electrically connected to the contact sections of the needle elements by means of electrical through-contacts in the shape of vias. The electronics device may be electrically contacted to the contact pads, for example by solder bumps placed on the electronics device or by gold stud bumps placed on the electronics device and in addition a solder paste or other conductive paste arranged on the contact pads on the substrate body. Herein, openings are formed in the body portion of the form element corresponding to the contact pads on the substrate body, such that the electronics device may be electrically contacted to the contact pads through the body portion of the form element.

In one embodiment, a second cover layer is formed to encapsulate the electronics device on the second side of the form element. The second cover layer beneficially is made from a thermoplastic material, for example a liquid crystal polymer (LCP) material, PEEK, PTFE, FEP or another thermoplastic biocompatible material. The second cover layer herein may be made up from different sub-layers, wherein an inner sub-layer may be made from a thermoplastic material having a comparatively low melting point, for example a melting point at a temperature in between 100° C. and 200° C. An outer sub-layer in contrast may have a melting point at a significantly higher temperature, for example a temperature larger than 200° C., for example larger than 250° C. The inner sub-layer in particular may serve to compensate for a height of the electronics device within the multi-electrode array device. For this, the inner sub-layer may comprise a rather low melting point such that it may easily flow and adapt to the shape of the electronics device. The second, outer sub-layer serves to cover the electronics device towards the outside and may comprise a higher melting point such that it bounds the material of the first, inner sub-layer towards the outside.

By placing a first cover layer on a first side of the substrate body and by placing the form element, the electronics device, and a second cover layer on the second side of the substrate body, a stack of layers is formed to fabricate the multi-electrode array device. In order to join the different layers together, heat may be applied to the stack formed by the different layers, in particular at a temperature beyond the melting point of a high-melting material as used for any one of the layers. For example, thermodes may be placed on a top and on a bottom of the stack and may be heated to a temperature for example between 250° C. and 350° C., for example between 265° C. and 320° C. at a pressure in between 0.01 bar to 2 bar for a time span between 10 seconds to 5 minutes, for example 30 seconds. The layers hence are heated and at the same time compressed, such that the layers are joined with one another and cavities within the stack are filled to form the multi-electrode array device with the needle sections of the needle elements protruding towards the outside and the electronics device encapsulated within.

The idea of the invention shall subsequently be described in more details with respect to the embodiments shown in the figures. Herein:

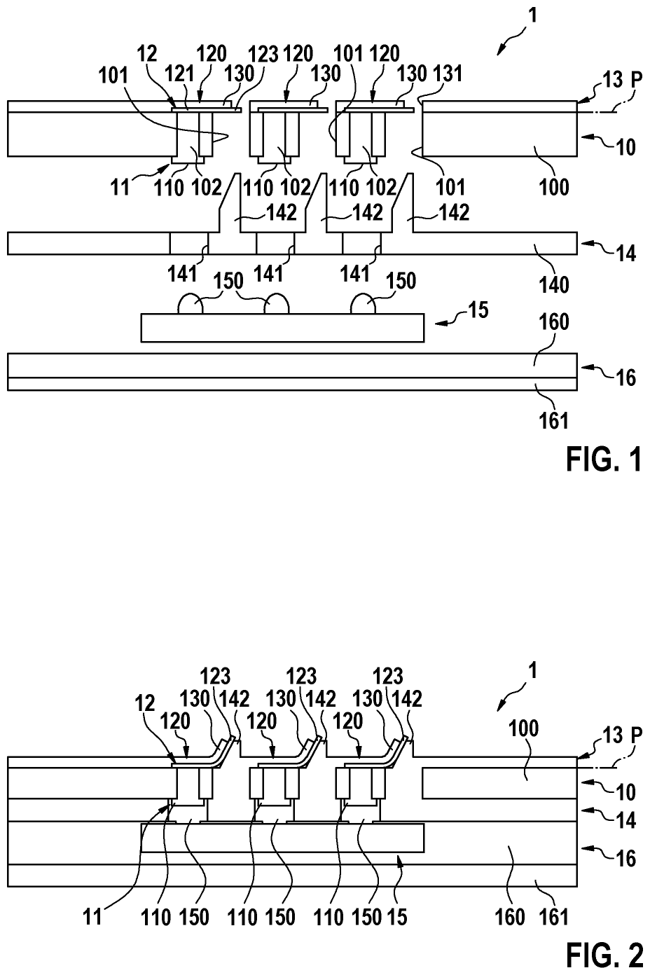

FIG. 1 shows a schematic drawing of a stack of layers of a multi-electrode array device;

FIG. 2 shows the stack of layers of FIG. 1, in a joined state;

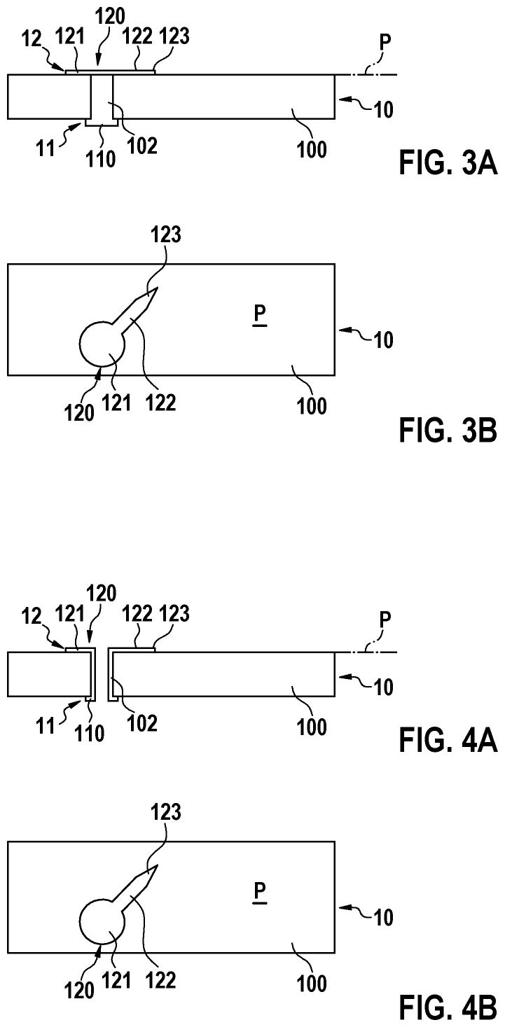

FIG. 3A shows a schematic cross-sectional drawing of a substrate with structured metal layers arranged thereon;

FIG. 3B shows a top view of the arrangement of FIG. 3A;

FIG. 4A shows a schematic cross-sectional drawing of a substrate with structured metal layers arranged thereon, according to another embodiment;

FIG. 4B shows a top view of the arrangement of FIG. 4A;

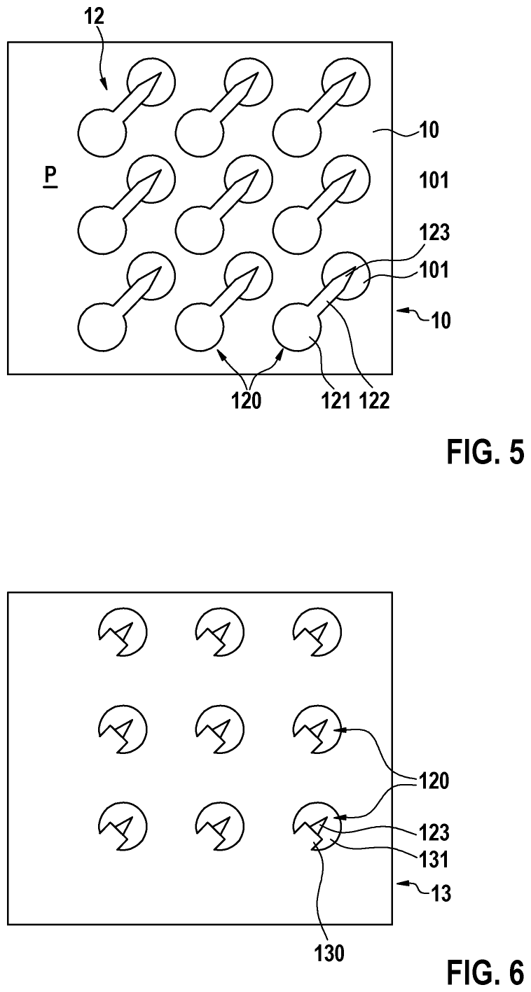

FIG. 5 shows a schematic top view of a substrate with multiple needle elements arranged thereon, in a pre-state prior to bending needle sections of the needle elements;

FIG. 6 shows a top view of FIG. 5, with a first cover layer arranged on the substrate;

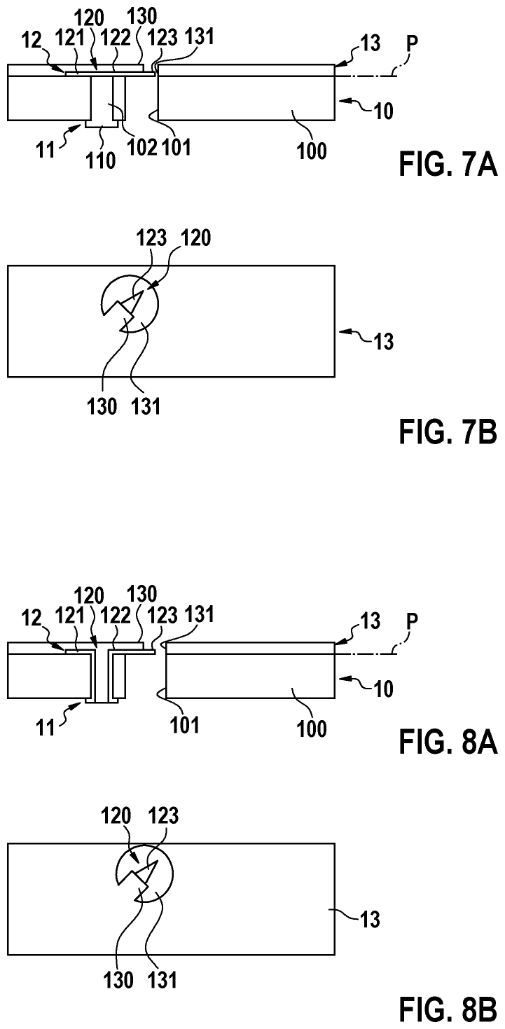

FIG. 7A shows a cross-sectional view through the substrate with the first cover layer arranged thereon, in the region of a needle element in the pre-state;

FIG. 7B shows a top view of the arrangement of FIG. 7A;

FIG. 8A shows a cross-sectional view through the substrate with the first cover layer arranged thereon, in the region of a needle element in the pre-state, according to another embodiment;

FIG. 8B shows the arrangement of FIG. 8A in a top view;

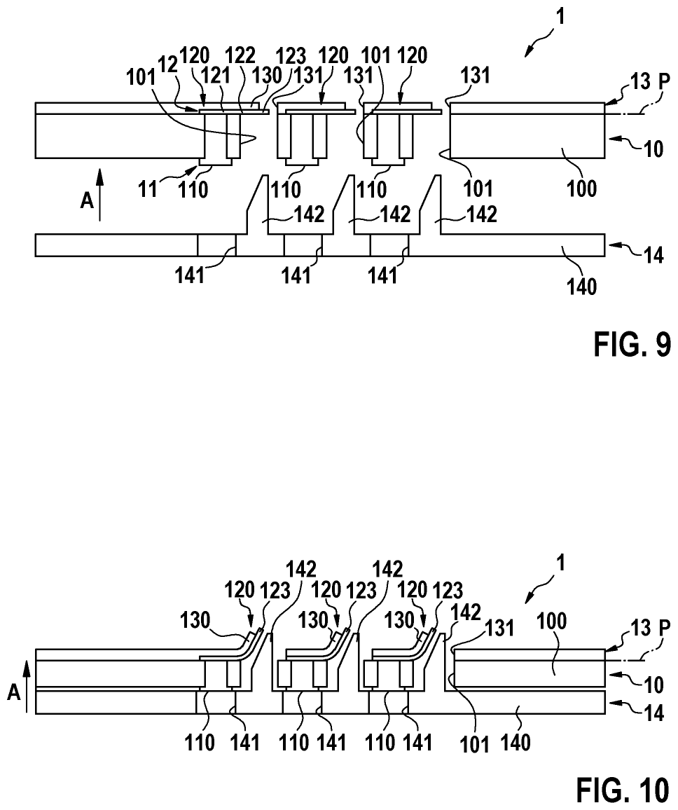

FIG. 9 shows the substrate with the first cover layer arranged thereon and an arrangement of needle elements in the pre-state prior to bending needle sections of the needle elements, together with a form element;

FIG. 10 shows the arrangement of FIG. 9, after placing the form element in a placement direction on the substrate for bending the needle sections;

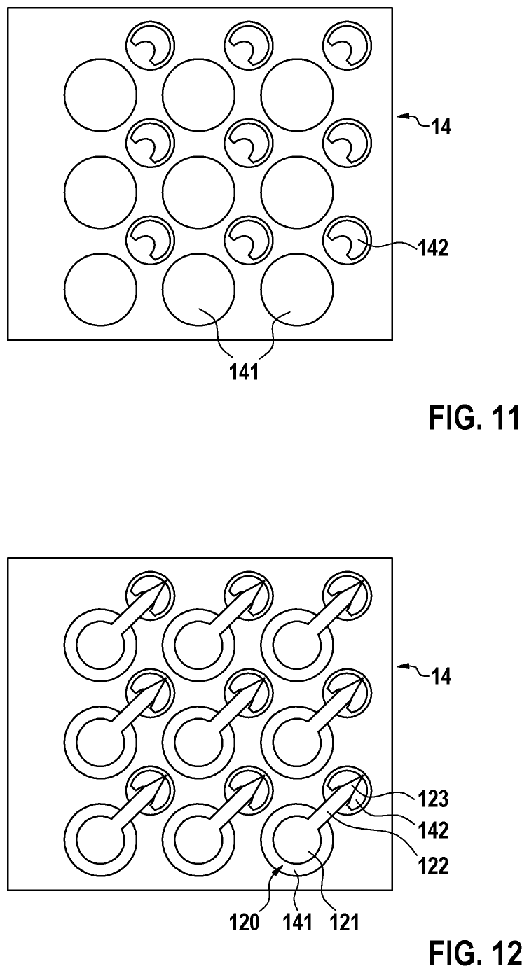

FIG. 11 shows a schematic top view of the form element;

FIG. 12 shows a schematic top view of the form element, with associated needle elements (in the pre-state) in an overlaid view;

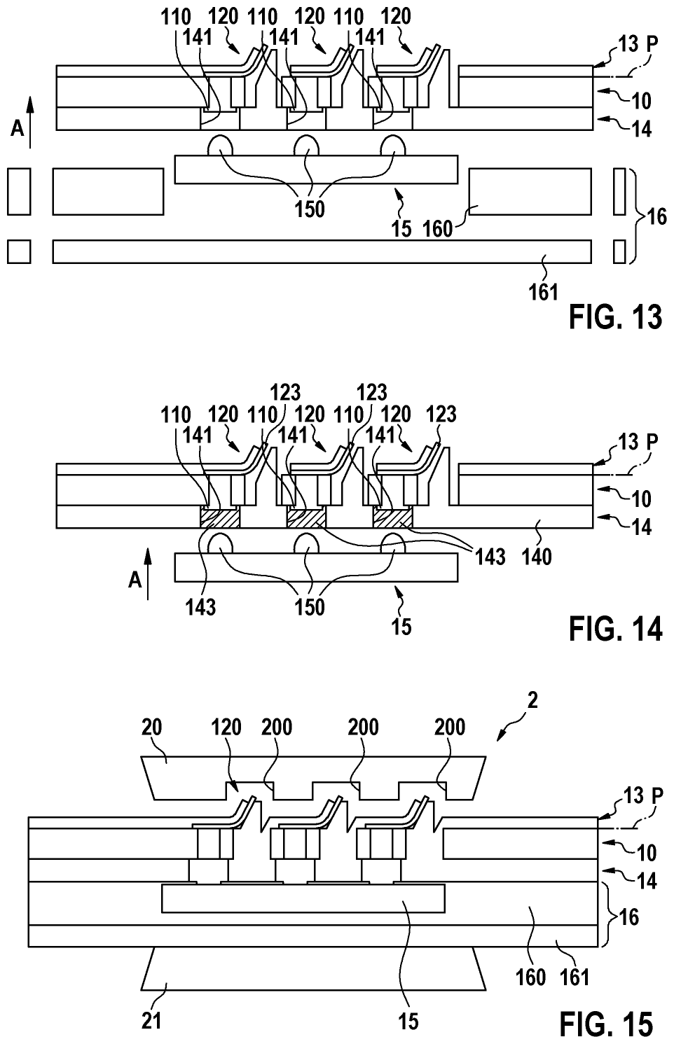

FIG. 13 shows the arrangement of FIG. 10, with an electronics device and a second cover layer for encapsulating the electronics device;

FIG. 14 shows the arrangement of FIG. 10, with an electronics device, according to another embodiment;

FIG. 15 shows the arrangement of FIG. 13, while applying a heating by means of thermodes to the stack of layers of FIG. 13;

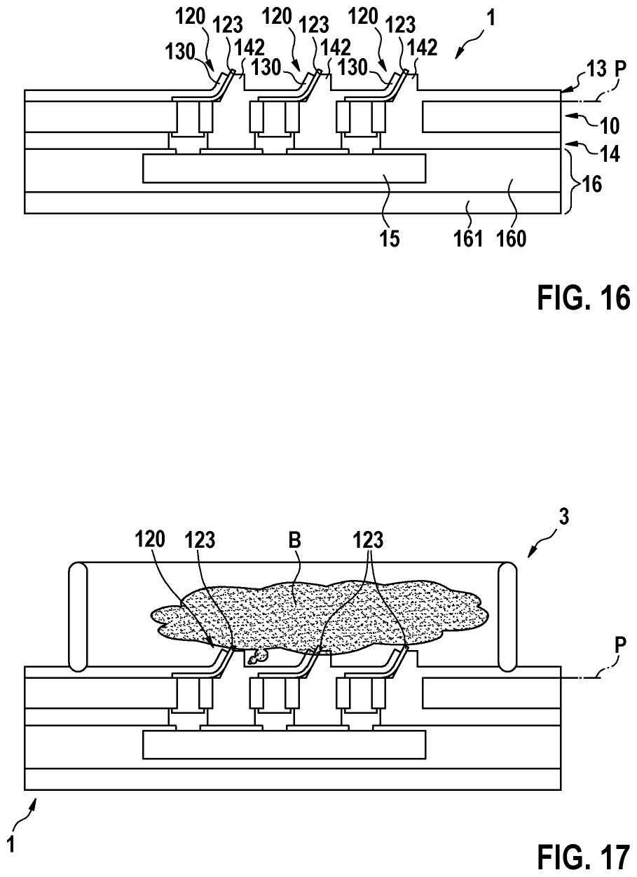

FIG. 16 shows a schematic cross-sectional view of the multi-electrode array device in a final state; and

FIG. 17 shows the multi-electrode array device during operation.

FIGS. 1 and 2 show an embodiment of a multi-electrode array device 1 formed by a stack of layers and comprising a multiplicity of electrodes formed by needle elements 120. FIG. 1 herein shows the stack of layers of the multi-electrode array device 1 prior to the forming of the multi-electrode array device 1, whereas FIG. 2 shows the multi-electrode array device 1 after fabrication in an operative state.

The multi-electrode array device 1 comprises a substrate 10 having a substrate body 100 on which two structured metal layers 11, 12 are formed. The metal layers 11, 12 are arranged on opposite sides of the substrate body 100, a metal layer 12 forming the needle elements 120 and a metal layer 11 forming contact pads 110 which are electrically contacted to contact portions 121 of the needle elements 120 by means of electrical vias 12.

The needle elements 120 are placed on a surface of the substrate body 100 to form an array of regularly or irregularly spaced electrodes. The needle elements 120 are spaced along a plane P, each needle element 120 forming a needle section 121 having a tip 123 protruding from the plane P and pointing towards the outside in order to engage with tissue during operation of the multi-electrode array device 1.

The substrate body 100 of the substrate 10 is made from a thermoplastic material, for example a liquid crystal polymer (LCP) material, PEEK, PTFE, FEP or another thermoplastic biocompatible material.

The substrate body 100 at the surface carrying the metal layer 12 forming the needle elements 120 is covered by a first cover layer 13 made of an electrically insulating material, in particular a thermoplastic material, for example a liquid crystal polymer (LCP) material, PEEK, PTFE, FEP or another thermoplastic biocompatible material. The first cover layer 13 may use the same material as the substrate body 100, or may be made from a different material. The first cover layer 13 covers the contact portions 121 of the needle elements 120 towards the outside and in addition covers a portion of each needle section 122 of the needle elements 120, as visible from FIG. 2, such that only a tip 123 of each needle element 120 is exposed towards the outside and may come into contact with tissue during operation of the multi-electrode array device 1.

The substrate body 100 may have a thickness (measured along a direction perpendicular to the plane P) in between 10 μm to 100 μm. The first cover layer 13 may have a thickness for example in between 10 μm to 100 μm.

The metal layer 12 forming the needle elements 120 may be made from gold and may have a thickness for example in between 1 μm and 50 μm. The metal layer 11 may be made from gold or a copper material and may have a thickness in between 1 μm and 50 μm. The vias 102 may be formed from a gold material or a copper material, for example by a gold plating or a copper plating.

Beneath the substrate body 100 a form element 14 is arranged, which by means of protrusion members 142 protruding from a body portion 140 reaches through openings 101 in the substrate body 100 such that the protrusion members 142 act onto the needle sections 122 of the needle elements 120 and together with the cover layer 13 cover the needle sections 122 such that only the tip 123 of the needle sections 122 of the needle elements 120 are exposed towards the outside, as it is visible from FIG. 2. As it shall be explained further below, the form element 14 during fabrication of the multi-electrode array device 1 serves to act onto the needle sections 122 of the needle elements 120 in order to bend the needle sections 122 with respect to the contact sections 121 for plastically deforming the needle sections 122 such that the needle sections 122 protrude from the substrate body 100 towards the outside and hence emerge from the plane P of the metal layer 12.

At a side of the form element 14 opposite to the substrate body 100, an electronics device 15, for example an ASIC chip, is arranged, which electrically contacts the contact pads 110 associated with the needle elements 120 through openings 141 in the body portion 140 of the form element 14. For this, the electronics device 15 comprises contact bumps 150, for example of a solder paste, which provide for an electrical contact in between the electronics device 15 and the contact pads 110, as visible from FIG. 2.

The electronics device 15 is encapsulated in the material of a second cover layer 16 made up of two sub-layers 160, 161. A first sub-layer 160 receives the electronics device 15 therein such that the electronics device 15 is embedded in the material of the first sub-layer 160. The first sub-layer 160 is covered towards the outside by a second sub-layer 161.

The form element 14 in particular may be provided as an injection molded part and beneficially is made of a thermoplastic material, for example a liquid crystal polymer (LCP) material, PEEK, PTFE, FEP or another thermoplastic biocompatible material. The material of the form element 14 beneficially has a comparatively high melting point, beneficially a melting point above a temperature of 200° C., for example above 250° C., in particular above 265° C.

Likewise, the second cover layer 16 embedding the electronics device 15 therein may be made of a thermoplastic material, for example a liquid crystal polymer (LCP) material, PEEK, PTFE, FEP or another thermoplastic biocompatible material. The first sub-layer 160 for this may for example be made from a material having a comparatively low melting point, for example in between 100° C. to 200° C. The first cover layer 160 is covered towards the outside by the second sub-layer 161 having a higher melting point, for example above 200° C., for example above 250° C., in particular above 265° C., such that the material of the first sub-layer 160 is confined towards the outside by means of the second sub-layer 161.

In an operative state, as shown in FIG. 2, the needle elements 120 are electrically contacted to the electronics device 15 embedded within the material of the different layers of the multi-electrode array device 1. Each needle element 120 herein with a needle section 122 protrudes towards the outside such that a tip 123 of each needle element 120 is exposed towards the outside and may come into contact with tissue, in particular brain tissue, during operation of the multi-electrode array device 1.

The electronics device 15 may in particular be a semiconductor device, such as a semiconductor chip, for example an ASIC chip. The electronics device 15 may provide for a preprocessing of signals received via the needle elements 120, for example an amplification, a digitization and a multiplexing of signals. The electronics device 15 may electrically be supplied with energy by a supply line reaching through the material of the cover layer 16, and may be in signal connection with an external device by a wire-bound connection or by a wireless connection.

The needle sections 122 of the needle elements 120 may each have a length between 10 μm to 100 μm, for example between 20 μm to 30 μm, a width between 1 μm and 50 μm, for example between 5 μm and 15 μm, and a thickness (corresponding to the thickness of the metal layer 12 on the substrate 10) between 1 μm and 50 μm, for example between 1 μm and 10 μm.

The needle sections 122 are bent with respect to the contact sections 121 of the needle elements 120, but are integrally formed from the metal layer 12 on the substrate body 100 of the substrate 10. The needle sections 122 are arranged at an angle with respect to the plane P of the surface of the substrate body 100, in particular an angle in between 45° to 90°, beneficially between 60° to 90°.

Subsequently, with reference to FIGS. 3A, 3B to 16, embodiments of fabrication of a multi-electrode array device 1 shall be explained.

Referring now to FIGS. 3A and 3B, for fabricating a multi-electrode array device 1 a substrate 10 having a substrate body 100 is provided, with metal layers 11, 12 being arranged on opposite surfaces of the substrate body 100. The metal layers 11, 12 are structured such that needle elements 120 are formed on a first side of the substrate body 100 and contact pads 110 are formed on an opposite, second side of the substrate body 100. Electrical vias 102 reach through the substrate body 100 to electrically contact the contact sections 121 of the needle sections 122 and associated contact pads 110.

As visible from the top view of FIG. 3B, each needle element 120 is formed by a structuring of the metal layer 12, for example by employing laser ablation, plasma etching or chemical etching, such that each needle element 120 comprises a contact section 121 having a circular disc shape and a needle section 122 extending from the contact section 121 and having a tip 123 at an end opposite to the contact section 121.

As the needle section 122 of each needle element 120 is formed from the metal layer 12 on the substrate body 100, the needle section 122 in an initial state extends along the plane P of the metal layer 12, such that the contact section 121 and the needle section 122 extend along a common plane.

In the embodiment of FIGS. 3A and 3B, the contact pads 110 at the side of the substrate body 100 opposite to the needle elements 120 is formed from a copper material. Likewise, the electrical vias 122 are formed from a copper material. The metal layer 12 forming the needle elements 120 is made from a gold material.

In another embodiment shown in FIGS. 4A and 4B, both the metal layer 12 forming the needle elements 120 at the first side of the substrate body 100 and the metal layer 11 forming the contact pads 110 at the second side of the substrate body 100 are formed from a gold material, and also the vias 102 in the substrate body 100 are formed from a gold material.

With respect to the shape of the needle elements 120, the embodiments of FIGS. 3A and 3B and FIGS. 4A and 4B are identical.

FIG. 5 shows a top view of the substrate 10 with an array of needle elements 120 formed thereon. Herein, in a subsequent step after structuring the gold layer 12, openings 101 are formed in the substrate body 100, the openings 101 being arranged such that the needle sections 122 each project into a space aligned with a corresponding opening 101 such that at least an end portion of each needle section 122 no longer is supported by the material of the substrate body 10 and freely extends across the associated opening 101.

Each opening 101 may for example have a width in between 0.1 to 10 mm. The openings 101 for example may be formed by laser ablation, plasma etching or chemical etching.

Following the forming of the openings 101 in the substrate body 100, the cover layer 13 is formed on the surface of the substrate body 100 carrying the needle elements 120, as visible from FIG. 6 and in addition from FIGS. 7A, 7B and FIGS. 8A, 8B, corresponding to the two different embodiments of FIGS. 3A, 3B and 4A, 4B. Openings 131 are formed in the cover layer 13 corresponding to the openings 101 in the substrate body 100, wherein a cover portion 130 remains on the needle sections 122 such that only the tip 123 of each needle section 122 is exposed.

The cover layer 13 beneficially is made from a photo-structurable polymer material, for example a photoresist, and is structured to form the openings 131 therein using for example an etching technique such as plasma etching or chemical etching.

Referring now to FIGS. 9 and 10, after forming the cover layer 13 on the substrate 10, a form element 14 is provided and placed along a placement direction A on the side of the substrate 10 opposite to the cover layer 13. The form element 14 beneficially is provided as an injection molded part, for example made from a thermoplastic material, the form element 14 comprising a body portion 140 and protrusion members 142 protruding from the body portion 140 to be inserted into the openings 101 of the substrate body 100 along the placement direction A.

Openings 141 are formed in the body portion 140 corresponding to the contact pads 110.

By inserting the protrusion members 142 into the openings 101 of the substrate body 100, the protrusion members 142 act onto the free portions of the needle sections 122 projecting into the space of the openings 101, as it is visible in the transition of FIG. 9 to FIG. 10. By acting onto the needle sections 122, the needle sections 122 are bent with respect to the contact sections 121, such that the needle sections 122 are deflected outwards, as visible from FIG. 10.

As visible from FIG. 11 showing a top view of the form element 14, the protrusion members 142 may have a cross-sectional shape of a semi-circular hollow cylinder. The protrusion members 142 hence receive the needle sections 122 therein such that the needle sections 122 are embedded in between the cover portions 130 of the cover layer 13 and the protrusion members 142 of the form element 14, as visible from FIG. 10 in view of FIG. 12 (showing the form element 14 with the corresponding needle elements 120 in an overlaid fashion).

By placing the form element 14 on the substrate 10, all needle sections 122 are bent and deflected with respect to the contact sections 121 at the same time in a single processing step. The plastic deformation of the needle sections 122 herein may be facilitated by heating the needle sections 122, for example by blowing hot air having a temperature for example in between 100° C. to 200° C. towards the surface of the substrate 10.

At the end of the bending step, the needle sections 122 point upwards with respect to the substrate 10 and are arranged with respect to the plane P at an angle for example in between 45° to 90°, beneficially between 60° to 90°, such that the needle sections 122 with their tips 123 point outwards and are exposed at their tips 123 towards the outside.

After placing the form element 14 on the substrate 10, the electronics device 15 is placed on the form element 14, as shown in FIGS. 13 and 14. The electronics device 15 in the embodiment of FIG. 13 comprises contact bumps 150 made from a solder paste material, whereas in the embodiment of FIG. 14 the electronics device 15 comprises contact bumps 150 made from a gold material, in which case a solder paste or solder glue 143 is introduced into the openings 141 of the body portion 140 of the form element 14 for example by using a screen print technique or a micro dispensing technique.

The contact bumps 150 are arranged on the electronics device 15 to correspond to the locations of the openings 141 in the body portion 140 of the form element 14 and hence to the locations of the contact pads 110 on the substrate 10, as visible from FIGS. 13 and 14.

In addition, as shown in FIG. 13, the second cover layer 16 is placed on the form element 14, the first sub-layer 160 of the cover layer 16 serving to compensate for a height of the electronics device 15 and the second sub-layer 161 serving to cover the first sub-layer 160 and the electronics device 15 towards the outside.

In a concluding step, as shown in FIG. 15, thermodes 20, 21 of a forming tool 2 are placed on either side of the stack of layers in order to apply a heat having a peak temperature in between for example 265° to 320° C. and a pressure in between 0.01 bar to 2 bar over a time span in between for example 10 seconds to 5 minutes to the stack of layers. An upper thermode 20 comprises cavities 200 receiving the protruding needle sections 122 therein such that the needle sections 122 are not deformed by the action of the thermodes 20, 21.

The layers of the multi-electrode array device 1 hence are joined with respect to each other and cavities within the stack are filled. In addition, a soldering connection in between the electronics device 15 and the contact pads 110 is established.

In an operative state, the needle sections 122 of the needle elements 120 with their tips 123 protrude towards the outside, and the electronics device 15 is electrically contacted to the contact pads 110 and hence to the needle elements 120. The electronics device 15 herein is fully received and embedded within the material of the cover layer 16 and hence is encapsulated within the different layers of the multi-electrode array device 1.

Referring now to FIG. 17, during operation of the multi-electrode array device 1 the tips 123 of the needle elements 120 may be brought into contact with tissue B, for example brain tissue, in in vivo or in vitro applications. As shown in FIG. 17, the multi-electrode array device 1 may for example be used in conjunction with a carrier device, for example a so-called lab-on-chip cartridge 3, such that the needle elements 120 electrically contact tissue received within the carrier device.

By means of the multi-electrode array device 1 neuronal action potentials may be recorded within tissue in in vivo or in vitro applications. Needle elements 120 herein are formed in a microscopic scale to form an array of electrodes to engage with neuronal cells to sense signal patterns of electrical potentials across tissue.

The idea of the invention is not limited to the embodiments described above, but may be implemented in an entirely different fashion.

A multi-electrode array device may comprise any number of needle elements forming electrodes, for example a number larger than 2, beneficially a number larger than 5, for example larger than 10.

The needle elements are formed by a structured metal layer on a substrate, allowing to fabricate the needle elements to have any desired shape while making fabrication easy, cost-efficient and reliable. Needle sections of the needle elements in particular may be formed to have a desired length for coming into contact with tissue.

### LIST OF REFERENCE NUMERALS

1 Multi-electrode array device
10 Substrate
100 Substrate body
101 Opening
102 Via
11 Metal layer
110 Contact pad
12 Metal layer
120 Needle element
121 Contact section
122 Needle section
123 Tip
13 Cover layer
130 Cover portion
131 Opening
14 Form element
140 Body portion
141 Opening
142 Protrusion member
143 Conductive paste material
15 Electronics device
150 Contact bumps
16 Cover layer
160 Low melting substrate
161 High melting substrate
2 Tool
20, 21 Thermode
200 Cavity
3 Lab-on-chip cartridge
A Placement direction
B Brain tissue
P Plane

1. A multi-electrode array device (1), comprising
a substrate (10) having a substrate body (100), and
a multiplicity of electrodes formed by a multiplicity of needle elements (120) arranged on said substrate body (100) and spaced with respect to each other along a plane (P),
characterized in that said needle elements (120) are formed from a metal layer (12) arranged on said substrate body (100), each needle element (120) comprising a contact section (121) extending along said plane (P) and a needle section (122) extending from said contact section (121) and having a tip (123), wherein said needle section (122) is bent with respect to said contact section (121) such that the needle section (122) with its tip (123) protrudes from said plane (P).

2. The multi-electrode array device (1) according to claim 1, characterized in that said substrate body (100) forms a surface extending along said plane (P), the needle elements (120) being arranged on said surface such that the contact sections (121) are placed on the surface and the needle sections (122) protrude from the surface.

3. The multi-electrode array device (1) according to claim 2, characterized by a first cover layer (13) covering said surface of the substrate body (100) and said contact sections (121) on the surface.

4. The multi-electrode array device (1) according to claim 1, characterized in that the substrate body (100) is formed from a thermoplastic material.

5. The multi-electrode array device (1) according to claim 1, further comprising a form element (14), wherein said needle elements (120) are arranged on a first side of the substrate body (100), and said form element (14) is arranged on a second side of the substrate body (100) opposite to the first side, said form element (14) comprising a multiplicity of protrusion members ( ) abutting the needle sections (122) of the multiplicity of needle elements (120).

6. The multi-electrode array device (1) according to claim 1, characterized by an electronics device (15), wherein said needle elements (120) are arranged on a first side of the substrate body (100) and a semiconductor device (15) is arranged on a second side of the substrate body (100) opposite to the first side, the electronics device (15) being electrically connected to the contact sections (121) of the needle elements (120) by an arrangement of electrical vias (102) extending through said substrate body (100).

7. The multi-electrode array device (1) according to claim 6, characterized in that the electronics device (15) is encapsulated within electrically insulating material of a second cover layer (16) on said second side of the substrate body (100).

8. A method for fabricating a multi-electrode array device (1), comprising
providing a substrate (10) having a substrate body (100), and
providing a multiplicity of electrodes formed by a multiplicity of needle elements (120) on said substrate body (100) such that the needle elements (120) are spaced with respect to each other along a plane (P),
characterized in that said providing said multiplicity of electrodes formed by said multiplicity of needle elements (120) includes: forming the needle elements (120) from a metal layer (12) arranged on said substrate body (100) such that each needle element (120) comprises a contact section (121) extending along said plane (P) and a needle section (122) extending from said contact section (121) and having a tip (123), wherein said needle section (122) is bent with respect to said contact section (121) such that the needle section (122) with its tip (123) protrudes from said plane (P).

9. The method according to claim 8, characterized in that the needle elements (120) are formed from the metal layer (12) by forming the contact sections (121) and the needle sections (122) to commonly extend along said plane (P) and to subsequently bent the needle sections (122) with respect to the contact sections (121) such that the needle sections (122) protrude from said plane (P).

10. The method according to claim 8, characterized in that the needle sections (122) are bent with respect to the contact sections (121) by placing a form element (14) on said substrate body (100), said form element (14) comprising a multiplicity of protrusion members (142) to act onto said needle sections (122) for bending the needle sections (122) with respect to the contact sections (121).

11. The method according to claim 10, characterized in that, prior to placing the form element (14) on the substrate body (100) for bending the needle sections (122), openings (101) are formed on the substrate body (100) such that each needle section (122) projects into a space aligned with a corresponding opening (101) and, for bending the needle sections (122) with respect to the contact sections (121), the form element (14) is placed on the substrate body (100) such that the protrusion members (142) are introduced into said openings (101) in said substrate body (100) to act onto said needle sections (122).

12. The method according to claim 10, characterized in that, prior to or after placing the form element (14) on the substrate body (100) for bending the needle sections (122), a first cover layer (13) is formed on the substrate body (100) to at least cover said contact sections (121) of the needle elements (120).

13. The method according to claim 10, characterized in that, for bending the needle sections (122), the form element (14) is placed on the substrate body (100) along a placement direction (A) such that the substrate body (100) is arranged on a first side of the form element (14).

14. The method according to claim 13, characterized in that, after placing the form element (14) on the substrate body (100) for bending the needle sections (122), an electronics device (15) is placed on a second side of the form element (14) and is electrically connected to the contact sections (121) of the needle elements (120).

15. The method according to claim 14, characterized in that a second cover layer (16) is formed to encapsulate said electronics device (15) on said second side of the form element (14).
